(19) United States
(12) Patent Application Publication
(10) Pub. No.: US 2022/0339020 A1
(43) Pub. Date: Oct. 27, 2022

(54) NOVEL DEVICE AND METHOD FOR THE TREATMENT OF TEMPOROMANDIBULAR JOINT DISORDER

(71) Applicant: Charles Sutera, III, Medford, MA (US)

(72) Inventor: Charles Sutera, III, Medford, MA (US)

(21) Appl. No.: 17/660,357

(22) Filed: Apr. 22, 2022

Related U.S. Application Data
(60) Provisional application No. 63/178,480, filed on Apr. 22, 2021.

(51) Int. Cl. A61F 5/058

(52) U.S. Cl. CPC A61F 5/05891

(57) ABSTRACT

A dental device for treating temporomandibular joint disorder and associated symptoms. The device is a plurality of oral appliances produced in sequence via data from bite registrations as bite position changes while progressing through wearing said sequence of appliances. The appliance may securely attach to a plurality of teeth and contains an occluding surface which contacts the opposing arch of teeth. The appliance equilibrates the occlusal contacts and at least reduces protrusive interferences and/or lateral excursive interferences of the occlusion. As a patient wears the appliances, the mandible may passively reposition into a more comfortable position. The device enables an improved method compared to existing methods for treatment of TMJ disorder and associated symptoms. Improved methods of manufacturing were designed using computer implemented methods of design and manufacturing, and a method for adjusting the mandibular position via of bite registrations or minor adjustments to the occlusion of the device.

### REFERENCE TO RELATED APPLICATIONS

This application claims priority to U.S. Patent Application No. 63/178,480, filed Apr. 22, 2021, and titled, “A NOVEL DEVICE AND METHOD FOR THE TREATMENT OF TEMPOROMANDIBULAR JOINT DISORDER,” the contents of which is incorporated by reference in its entirety.

### BACKGROUND

Temporomandibular joint disorder is a condition which causes a multitude of symptoms of the head and neck included but not limited to muscle tension, headaches, and bruxism. The mandibular position at which the temporomandibular complex routinely functions is a primary etiology of said symptoms.

Oral splint therapy has been supported by literature to effectively treat symptoms of temporomandibular joint disorder. There are currently oral splint devices which are cleared to treat muscle tension and headaches associated with TMJ disorder. However, the devices available do not include all active features supported by literature to be effective, the devices require dentists to complete significant chairside protocols, and the devices available do not utilize novel techniques of computer-aided design and additive manufacturing.

### BRIEF DESCRIPTION OF THE DRAWINGS

The features and advantages of the present disclosure will be more fully understood with reference to the following detailed description when taken in conjunction with the accompanying figures, wherein:

FIG. 1 is an oblique view of a device demonstrating a preferred embodiment comprising a single or a plurality of mandibular position adjustment appliances having a polymeric shell with cavities shaped to receive teeth.

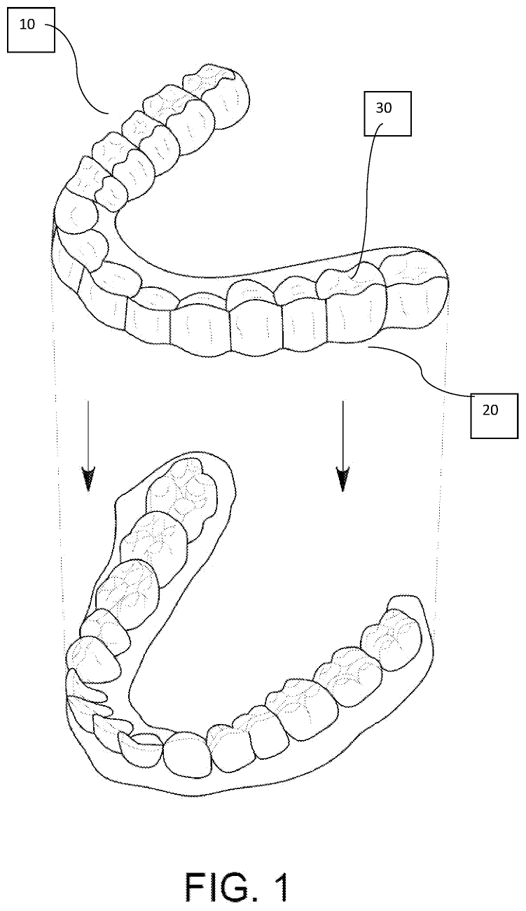

FIG. 2 is a frontal view of a device demonstrating a preferred embodiment comprising an occlusal surface which does not contact the non-supporting cusps of the opposing arch, and a means to at least reduce occlusal interferences from lateral excursive movement and/or protrusive movement.

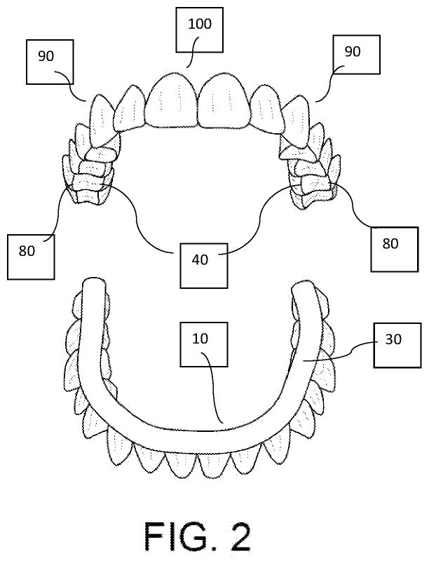

FIG. 3 is a frontal view of a device and the mandible in lateral excursion demonstrating a preferred embodiment comprising a means to at least reduce occlusal interferences from lateral excursive movement and/or protrusive movement.

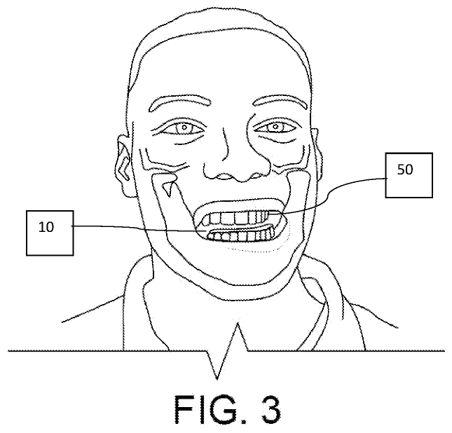

FIG. 4 is a profile view of a device and the mandible in protrusive excursion demonstrating a preferred embodiment comprising a means to at least reduce occlusal interferences from lateral excursive movement and/or protrusive movement.

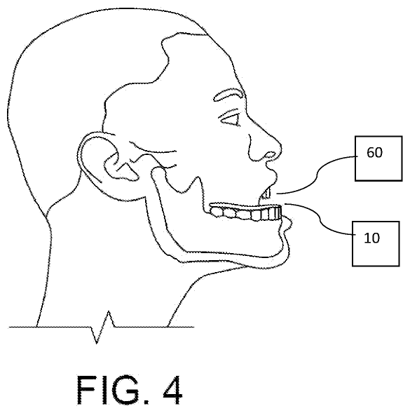

FIG. 5 is a frontal view of a device and the mandible in a comfortable position compared to the mandible in centric occlusion without the device demonstrating a preferred embodiment comprising a means of passively repositioning the mandible and at least one appliance which is shaped to provide traction from the occlusal surface to the opposing teeth such that the resulting mandibular position when worn is more comfortable than the mandibular position when the teeth are in centric occlusion.

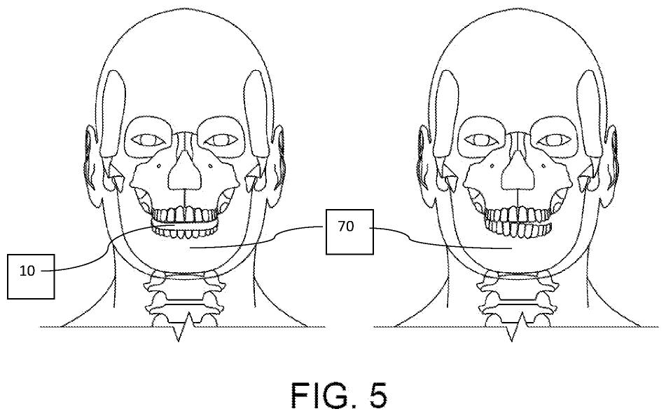

FIG. 6 is a profile view of a device and the mandible in protrusive excursion demonstrating a preferred embodiment comprising specifying one or a sequence of bite registrations to manufacture device such that said device repositions the mandible to a more comfortable position, wherein at least some of the bite registrations are represented by digital data sets, wherein specifying a sequence of bite registrations comprises moving the mandible according to an optimization function; and producing one or more appliances in accordance with the digital data sets.

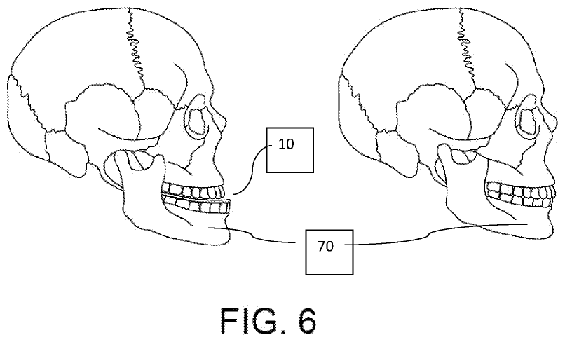

### SUMMARY

In one aspect, the present disclosure provides an oral appliance for repositioning the mandible of a user. In particular, the disclosed apparatus provides a first polymeric shell having an occlusal surface, an outer wall, and an inner wall that define a plurality of cavities shaped to receive a tooth arch of the user, the occlusal surface configured to contact a supporting cusp of a tooth arch in opposition to the tooth arch of the user, and wherein the occlusal surface is configured to not come into contact with any of the non-supporting cusps, canine teeth, or anterior teeth of the tooth arch in opposition to the tooth arch of the user.

In another aspect, the disclosed apparatus provides at least one polymeric shell has been fabricated using patient data comprising a sequence of bite registrations indicating an optimally comfortable position.

In yet another aspect, the polymeric shell has been fabricated using a digital data set representing a tooth interarch position that is acceptable to the user. In a further aspect, the polymeric shell is configured to minimize lateral excursive interferences. In a further aspect, the first polymeric shell may be configured to minimize protrusive interferences.

In another aspect, the occlusal surface may be further configured to equilibrate the distribution of bite force on the tooth arch of the user. In yet another aspect, the present disclosure provides an apparatus comprising a second polymeric shell having an occlusal surface, an outer wall, and an inner wall that define a plurality of cavities, which second polymeric shell is configured to fit with the first polymeric shell in the mouth of a user.

In a further aspect, the present disclosure provides a method for fabricating an oral appliance for repositioning the mandible of a user, comprising executing on a processor the steps of receiving, on a computer, a digital bite registration data indicating the position of at least on tooth arch of the user; storing the digital bite registration data in the memory of the computer; using the stored digital bite registration data to generate a digital arrangement of the user's teeth; applying an optimization function to the digital arrangement of the user's teeth to generate an optimized production data set; and transmitting the optimized production data set for fabrication of mandibular adjustment device having a first polymeric shell having an occlusal surface, an outer wall, and an inner wall that define a plurality of cavities shaped to receive a tooth arch of the user, wherein the occlusal surface is formed according to the optimized production data set.

### DETAILED DESCRIPTION

In embodiments of the invention, a mandibular repositioning device is provided for repositioning the mandible into a position that reduces headaches and tension of the muscles of the head and neck by attachment to either the maxillary or mandibular teeth. Said device has an occlusal surface which contacts the opposing arch upon biting so that occlusal contact is only on supporting cusps, occlusal force on supporting cusps is distributed evenly, and lateral excursive and protrusive interferences are reduced and/or fully removed.

In embodiments, a single or a plurality of mandibular position adjustment appliances may have a polymeric shell with cavities shaped to receive teeth, an occlusal surface which when worn by a patient has means to equilibrate distribution of bite force and a means to at least reduce lateral excursive and/or protrusive interferences of occlusion, a means of attaching to a plurality of teeth, and a means of repositioning the mandible whereby a patient may utilize the device to treat headaches and muscle tension of the head and/or neck and may prevent bruxism and TMJ disorder.

In embodiments, a device may comprise a single or a plurality of mandibular position adjustment appliances having a polymeric shell with cavities shaped to receive teeth, a means of attaching to a plurality of teeth, an occlusal surface which does not contact the non-supporting cusps of the opposing arch, a means to at least reduce occlusal interferences from lateral excursive movement and/or protrusive movement whereby a patient may utilize the device to treat headaches and muscle tension of the head and/or neck and may prevent bruxism and TMJ disorder.

A device may function in an embodiment comprising a single or a plurality of mandibular position adjustment appliances produced in a series having a polymeric shell with cavities shaped to receive teeth, a means of attaching to a plurality of teeth, an occlusal surface which does contact the supporting cusps of the opposing arch, does not contact the non-supporting cusps of the opposing arch, does not contact the canine teeth of the opposing arch, and does not contact the anterior teeth of the opposing arch whereby a patient may utilize the device to treat headaches and muscle tension of the head and/or neck and may prevent bruxism and TMJ disorder.

In embodiments, a method is disclosed for designing a device that reduces headaches and tension of the muscles of the head and neck by utilizing a computer aided design algorithm. In said computer aided design algorithm the device is designed by equilibrating the occlusal surface such that occlusal contact is only on supporting cusps, occlusal force on supporting cusps is distributed evenly, and lateral excursive and protrusive interferences are removed. In embodiments, the mandible of a user is repositioned by the specification of a sequence of bite registrations and appliances, the recording of a plurality of patient bite registrations, the fabrication of a plurality of oral appliances based on said bite registration data such that said devices progressively reposition the mandible as the devices are worn in sequence.

In embodiments, a method is described for repositioning the mandible by choosing at least one arch of teeth, placing an oral appliance on said arch of teeth that balances the bite force and reduces the lateral and protrusive interferences during excursive movements of the mandible, and creating means of passive rotation and/or translation of the mandible toward a desired position such that symptoms of muscle tension, headaches, bruxism, and/or tinnitus are reduced.

In embodiments, a computer-implemented method is described for producing appliances to treat symptoms of temporomandibular joint disorder which includes specifying one or a sequence of bite registrations to reposition the mandible to a more comfortable position, wherein at least some of the bite registrations are represented by digital data sets, wherein specifying a sequence of bite registrations comprises moving the mandible according to an optimization function; and producing one or more appliances in accordance with the digital data sets wherein the appliances comprise polymeric shells having cavities to receive teeth and an occlusal surface which equilibrates distribution of bite force and reduces and/or eliminates lateral excursive and/or protrusive interferences of occlusion

In an embodiment of a system for repositioning the mandible from an initial position to a desired position, said system comprising a single or a plurality of mandibular position adjustment appliances having a polymeric shell with cavities shaped to receive teeth, including at least one appliance which is shaped to provide traction from the occlusal surface to the opposing teeth such that the resulting mandibular position when worn is more comfortable than the mandibular position when the teeth are in centric occlusion.

A computer-implemented method in an embodiment for producing appliances to treat symptoms of temporomandibular joint disorder, comprising specifying one or a sequence of bite registrations to reposition the mandible to a more comfortable position, wherein at least some of the bite registrations are represented by digital data sets, wherein specifying a sequence of bite registrations comprises moving the mandible according to an optimization function; and producing one or more appliances in accordance with the digital data sets wherein the appliances comprise polymeric shells having cavities to receive teeth and an occlusal surface which equilibrates distribution of bite force and reduces and/or eliminates lateral excursive and/or protrusive interferences of occlusion, wherein specifying a sequence of bite registrations comprises recording bite registrations, fabricating appliances, and patient wearing said appliances until the sum of differential distances for the plurality of bite registrations exceeds a predetermined threshold, thereby producing a final digital data set.

A computer-implemented method in an embodiment for producing appliances to treat symptoms of temporomandibular joint disorder, comprising specifying one or a sequence of bite registrations to reposition the mandible to a more comfortable position, wherein at least some of the bite registrations are represented by digital data sets, wherein specifying a sequence of bite registrations comprises moving the mandible according to an optimization function; and producing one or more appliances in accordance with the digital data sets wherein the appliances comprise polymeric shells having cavities to receive teeth and an occlusal surface which equilibrates distribution of bite force and reduces and/or eliminates lateral excursive and/or protrusive interferences of occlusion, wherein specifying the sequence of bite registrations to reposition the mandible comprises: generating an initial digital data set representing an initial tooth interarch position; based on the initial digital data set, generating a mandibular positioning appliance which the patient wears for a period of time; and, generating a second data set representing a second tooth interarch position; and based on the second data set, generating a mandibular positioning appliance which the patient wears for a period of time

A computer-implemented method in an embodiment for producing appliances to reposition the mandible, comprising specifying a sequence of bite registration to move the mandible passively into a more comfortable position, wherein at least some of the bite registrations are represented by digital data sets; generating subsequent digital data sets based on prior digital data sets until a final digital data set representing an acceptable tooth interarch position is achieved; and producing one or more mandibular position adjustment appliances in accordance with the digital data sets wherein the appliances comprise a polymeric shell with cavities shaped to receive teeth, and an occlusal surface which has means to equilibrate distribution of bite force and a means to reduce lateral excursive and/or protrusive interferences of occlusion whereby the mandible repositions from one interarch position to a successive interarch position.

An apparatus in an embodiment for producing appliances to treat symptoms of temporomandibular joint disorder, the apparatus comprising means for generating a digital arrangement of teeth; means for specifying a sequence of bite registrations to an optimization function; and means for generating one or more appliances in accordance with the specified sequence of bite registrations, the appliances comprise a polymeric shell with cavities shaped to receive teeth, and an occlusal surface which has means to equilibrate distribution of bite force and a means to reduce lateral excursive and/or protrusive interferences of occlusion and reposition the mandible from one interarch position to a successive interarch position.

A computer program product in an embodiment of tangibly stored on a computer- readable medium, for producing appliances to treat symptoms of temporomandibular joint disorder, comprising instructions operable to cause a programmable processor to generate a digital arrangement of teeth; specify a single or a sequence of bite registrations to move the mandible to a more comfortable position; and generate one or more appliances in accordance with the specified sequence of tooth movements, the appliances comprise a polymeric shell with cavities shaped to receive teeth, and an occlusal surface which has means to equilibrate distribution of bite force and a means to reduce lateral excursive and/or protrusive interferences of occlusion and reposition the mandible from one interarch position to a successive interarch position.

The preferred embodiment is the attachment of a single or a plurality of devices to a plurality of teeth and is inserted by hand. The device may have an occlusal surface which contacts the opposing arch so that occlusal contact is evenly distributed only on supporting cusps and/or protrusive interferences of occlusion such that said reduction comprises a means to passively reposition the mandible over time such that it reduces the symptoms of temporomandibular joint disorder. The device does not contact on non-supporting cusps and reduces or eliminates contact on the opposing canine teeth and/or anterior incisors in the patient's more relaxed present bite position. The device may be manufactured in an embodiment that is customized to a single patient (e.g., plastic or thermoplastic acrylic resin) and may utilize advanced manufacturing (e.g., additive manufacturing). The device may be specified as a single device or as a plurality of devise in a series such that said device accommodates a new bite position as the mandible and musculature progressively repositions through use of previous devices in said series.

In an embodiment, a method is disclosed for designing a device that reduces headaches and tension of the muscles of the head and neck by utilizing a computer aided design algorithm. In said computer aided design algorithm the device is designed by equilibrating the occlusal surface such that occlusal contact is only on supporting cusps and protrusive interferences are removed A method functions in the preferred embodiment of repositioning the mandible by the specification of a sequence of bite registrations and appliances, the recording of a plurality of patient bite registrations, the fabrication of a plurality of oral appliances based on said bite registration data such that said devices progressively reposition the mandible as the devices are worn in sequence A method functions in the preferred embodiment of repositioning the mandible by choosing at least one arch of teeth, placing an oral appliance on said arch of teeth that balances the bite force and reduces the lateral and protrusive interferences during excursive movements of the mandible, and creating means of passive rotation and/or translation of the mandible toward a desired position such that symptoms of muscle tension, headaches, bruxism, and/or tinnitus are reduced.

In an embodiment, a method is disclosed for producing appliances to treat symptoms of temporomandibular joint disorder which includes specifying one or a sequence of bite registrations to reposition the mandible to a more comfortable position and/or protrusive interferences of occlusion

In an embodiment a system for repositioning the mandible from an initial position to a desired position is described, said system comprising a single or a plurality of mandibular position adjustment appliances having a polymeric shell with cavities shaped to receive teeth A computer-implemented method in the preferred embodiment for producing appliances to treat symptoms of temporomandibular joint disorder, comprising specifying one or a sequence of bite registrations to reposition the mandible to a more comfortable position, wherein at least some of the bite registrations are represented by digital data sets, wherein specifying a sequence of bite registrations comprises moving the mandible according to an optimization function and an occlusal surface which equilibrates distribution of bite force and reduces and/or eliminates lateral excursive and/or protrusive interferences of occlusion A computer-implemented method in the preferred embodiment for producing appliances to treat symptoms of temporomandibular joint disorder, comprising specifying one or a sequence of bite registrations to reposition the mandible to a more comfortable position, wherein at least some of the bite registrations are represented by digital data sets, wherein specifying a sequence of bite registrations comprises moving the mandible according to an optimization function and/or protrusive interferences of occlusion which the patient wears for a period of time

A computer-implemented method is described for producing appliances to reposition the mandible, comprising specifying a sequence of bite registration to move the mandible passively into a more comfortable position, wherein at least some of the bite registrations are represented by digital data sets; generating subsequent digital data sets based on prior digital data sets until a final digital data set representing an acceptable tooth interarch position is achieved; and producing one or more mandibular position adjustment appliances in accordance with the digital data sets wherein the appliances comprise a polymeric shell with cavities shaped to receive teeth and/or protrusive interferences of occlusion whereby the mandible repositions from one interarch position to a successive interarch position.

An apparatus is disclosed to treat symptoms of temporomandibular joint disorder, the apparatus comprising means for generating a digital arrangement of teeth; means for specifying a sequence of bite registrations to an optimization function; and means for generating one or more appliances in accordance with the specified sequence of bite registrations, the appliances comprise a polymeric shell with cavities shaped to receive teeth and/or protrusive interferences of occlusion and reposition the mandible from one interarch position to a successive interarch position.

A computer program product in an embodiment of tangibly stored on a computer-readable medium, for producing appliances to treat symptoms of temporomandibular joint disorder, comprising instructions operable to cause a programmable processor to generate a digital arrangement of teeth; specify a single or a sequence of bite registrations to move the mandible to a more comfortable position; and generate one or more appliances in accordance with the specified sequence of tooth movements, the appliances comprise a polymeric shell with cavities shaped to receive teeth and/or protrusive interferences of occlusion and reposition the mandible from one interarch position to a successive interarch position and/or protrusive interferences of occlusion and reposition the mandible from one interarch position to a successive interarch position [70].

It will be understood that there are numerous modifications of the illustrated embodiments described above which will be readily apparent to one skilled in the art, including any combinations of features disclosed herein that are individually disclosed or claimed herein, explicitly including additional combinations of such features. These modifications and/or combinations fall within the art to which this invention relates and are intended to be within the scope of the claims, which follow. It is noted, as is conventional, the use of a singular element in a claim is intended to cover one or more of such an element.

We claim:

1. An oral appliance for repositioning the mandible of a user comprising:
a first polymeric shell having an occlusal surface, an outer wall, and an inner wall that define a plurality of cavities shaped to receive a tooth arch of the user;
wherein the occlusal surface is configured to contact a supporting cusp of a tooth arch in opposition to the tooth arch of the user;
wherein the occlusal surface is configured to not come into contact with any of the non-supporting cusps, canine teeth, or anterior teeth of the tooth arch in opposition to the tooth arch of the user.

2. The oral appliance of claim 1 wherein the at least one polymeric shell has been fabricated using patient data comprising at least one registration indicating an optimally comfortable position.

3. The oral appliance of claim 1 wherein the at least one polymeric shell has been fabricated using a digital data set representing a tooth interarch position that is acceptable to the user.

4. The oral appliance of claim 1 wherein the occlusal surface is further configured to equilibrate the distribution of bite force on the tooth arch of the user.

5. The oral appliance of claim 1 further wherein the first polymeric shell is configured to minimize lateral excursive interferences.

6. The oral appliance of claim 1 further wherein the first polymeric shell is configured to minimize protrusive interferences.

7. The oral appliance of claim 1 further comprising a second polymeric shell having an occlusal surface, an outer wall, and an inner wall that define a plurality of cavities, which second polymeric shell is configured to replace the first polymeric shell in the mouth of a user.

8. The oral appliance of claim 1 further comprising a second polymeric shell having an occlusal surface, an outer wall, and an inner wall that define a plurality of cavities, which second polymeric shell is configured to fit with the first polymeric shell in the mouth of a user.

9. A computer-implemented method for fabricating an oral appliance for repositioning the mandible of a user, comprising executing on a processor the steps of:
receiving, on a computer, a digital bite registration data indicating the position of at least one tooth arch of the user;
storing the digital bite registration data in the memory of the computer;
using the stored digital bite registration data to generate a digital arrangement of the user's teeth;
applying an optimization function to the digital arrangement of the user's teeth to generate an optimized production data set; and
transmitting the optimized production data set for fabrication of mandibular adjustment device having a first polymeric shell having an occlusal surface, an outer wall, and an inner wall that define a plurality of cavities shaped to receive a tooth arch of the user, wherein the occlusal surface is formed according to the optimized production data set.

10. A computer-implemented method for producing appliances to treat symptoms of temporomandibular joint disorder comprising the steps of:
specifying a disoccluded bite registration to reposition the mandible to a more comfortable position according to an optimization function, the bite registrations being represented by digital data sets; and
producing one or more appliances in accordance with the digital data sets wherein the appliances comprise a polymeric shell having an occlusal surface, an outer wall, and an inner wall that define a plurality of cavities shaped to receive a tooth arch of the user.
